(19) United States
(12) Patent Application Publication
(10) Pub. No.: US 2026/0035651 A1
(43) Pub. Date: Feb. 5, 2026

(54) APPARATUS, SYSTEM, AND METHOD FOR REDUCING A MINIMUM WORKING VOLUME OF A BIOREACTOR

(71) Applicant: Global Life Sciences Solutions USA LLC, Marlborough, MA (US)

(72) Inventors: Nagaraj RAO, Bengaluru (IN); Thomas SMELTZER, Marlborough, MA (US); Milind MATHAPATI, Bengaluru (IN); Heather TRACY BEEM, Marlborough, MA (US); Kandakumar MURUGESAN, Bengaluru (IN); Saravanan BALAKRISHNAN, Bengaluru (IN); Subhanjan CHATTOPADHYAY, Bengaluru (IN)

(73) Assignee: Global Life Sciences Solutions USA LLC, Marlborough, MA (US)

(21) Appl. No.: 18/997,826

(22) PCT Filed: Jul. 26, 2023
(86) PCT No.: PCT/EP2023/070705

§ 371 Date: Jan. 23, 2025

(30) Foreign Application Priority Data
Aug. 5, 2022 (IN) 202211044844

(51) Int. Cl. C12M 1/34; C12M 1/00; C12M 1/06; C12M 3/00

(52) U.S. Cl. CPC C12M 41/44; C12M 23/14; C12M 23/48; C12M 27/06

(57) ABSTRACT

An apparatus for reducing a minimum working volume of a stirred tank includes a body having upper and lower surfaces, and an exterior profile shaped to closely fit within an interior of the stirred tank. The body including an interior portion extending through the body from the upper surface to the lower surface, the interior portion configured to receive and partially surround an impeller within the stirred tank, while allowing rotation of the impeller to agitate a fluid in the vessel when in use. The body further including an opening in a side surface of the body, the opening extending into the interior portion and configured to facilitate removal of fluid from the vessel when the stirred tank is in use, and to be removed from the stirred tank via a drain.

### CROSS-REFERENCE TO RELATED APPLICATIONS

This application is a national stage of International Application No. PCT/EP2023/070705 filed on Jul. 26, 2023, which claims the benefit of India application No. 202211044844, filed on Aug. 5, 2022, all of which are hereby incorporated by reference herein in their entireties.

### TECHNICAL FIELD OF THE INVENTION

Embodiments of the invention relate generally to mixer and bioreactor systems and methods, and, more particularly, to an apparatus, system, and method for selectively reducing a minimum working volume of a stirred tank mixer/bioreactor.

### BACKGROUND

Mixers and bioreactors are often employed to carry out biochemical and biological processes and/or manipulate liquids and other products of such processes. These devices typically utilize single use vessels e.g., flexible or collapsible plastic bags that are supported by an outer rigid structure such as a stainless-steel housing or tank. As will be appreciated, use of sterilized single use bags eliminates the time-consuming step of cleaning the tank after each use and reduces the chance of contamination.

In use, a disposable bag is positioned within the rigid tank and filled with the desired fluid for mixing/processing. An impeller assembly that includes a rotating impeller having one or more blades is disposed within the bag and is used to stir or mix the fluid. Existing impeller systems are either top-driven, having a shaft that extends downwardly into the bag, on which one or more impellers are mounted, or bottom-driven, having an impeller disposed in the bottom of the bag that is driven by, for example, a magnetic drive system positioned outside the bag and/or vessel.

Such mixers/bioreactors have minimum and maximum working volumes, i.e., the operational fluid volume range by which the device can effectively carry out biological/biochemical processes. The ratio of the maximum working volume to minimum working volume is referred to as the turndown ratio. For example, in current systems, a minimum working volume may approximately 33% of the maximum, for a turn down ratio of about 1:3. As will be appreciated, however, operational flexibility is highly desirable and the higher the turndown ratio, the greater range of acceptable fluid volumes.

More specifically, biologics are of high value, so it is desirable to manufacture only what is needed and minimize waste. However, known mixers and bioreactors operate with fixed turndown ratios and larger tanks/housings may have minimum working volumes that are not suitable for mixing/processing lower volumes. If a reactor or mixer has a low turndown ratio, extra biological materials may be necessary, e.g., a large inoculum train, and/or multiple reactors/mixers may be required. As will be appreciated, additional reactors/mixers requires additional bags, larger facilities, and generally increases operating costs.

In view of the above, there is a need for an apparatus, system, and method for selectively reducing a minimum working volume of a mixer/bioreactor.

### SUMMARY

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

In an embodiment, an apparatus for reducing a minimum working volume of a stirred tank includes a body having upper and lower surfaces, and an exterior profile shaped to closely fit within an interior of the stirred tank. The body including an interior portion extending through the body from the upper surface to the lower surface, the interior portion configured to receive and partially surround an impeller within the stirred tank, while allowing rotation of the impeller to agitate a fluid in the vessel when in use. The body further including an opening in a side surface of the body, the opening extending into the interior portion and configured to allow fluid to exit the body when the stirred tank is in use, and to be removed from the stirred tank via a drain.

In another embodiment, a stirred tank includes an interior volume configured to receive a vessel, the vessel having a vessel interior containing a rotatable impeller, the vessel interior configured to receive a fluid. The stirred tank further includes a body located within the interior volume of the stirred tank, the body having upper and lower surfaces and an interior portion extending through the body, the interior portion configured to receive and partially surround the impeller of a vessel placed within the stirred tank, while allowing rotation of the impeller to agitate fluid in the stirred tank when in use, the body further including an opening in a side surface of the body, the opening extending into the interior portion and configured to allow fluid to partially exit the interior portion when the stirred tank is in use, and to be removed from the vessel via a drain in the stirred tank. The body may be selectively removed from or added to the interior volume to increase or decrease a minimum working volume of the stirred tank.

In yet another embodiment, a method of reducing a minimum working volume in a stirred tank includes placing a body within an interior of the stirred tank, the body having upper and lower surfaces and an interior portion extending through the body, the interior portion configured to receive and partially surround an impeller of a vessel placed within the stirred tank, while allowing rotation of the impeller to agitate fluid in the stirred tank when in use; the body further including an opening in a side surface of the body, the opening extending into the interior portion and configured to allow fluid to partially exit the interior portion when in use, and to be removed from the vessel via a drain in the stirred tank.

In yet another embodiment, a method of increasing a minimum working volume in a stirred tank includes removing a body from an interior of the stirred tank, the body having upper and lower surfaces and an interior portion extending through the body, the interior portion configured to receive and partially surround an impeller of a vessel placed within the stirred tank, while allowing rotation of the impeller to agitate fluid in the stirred tank when in use;. The body further including an opening in a side surface of the body, the opening extending into the interior portion and configured to allow fluid to partially exit the interior portion when in use, and to be removed from the vessel via a drain in the stirred tank.

### BRIEF DESCRIPTION OF THE DRAWINGS

The present invention will be better understood from reading the following description of non-limiting embodiments, with reference to the attached drawings, wherein below:

FIG. 1 is a front perspective view of an exemplary mixer system suitable for use with embodiments of the invention.

FIG. 2 is a top perspective view of an interior of a stirred tank suitable for use with embodiments of the invention.

FIG. 3 is a front perspective view of a stirred tank cut away to illustrate an apparatus for reducing a minimum working volume according to an embodiment of the invention.

FIG. 4 is a top perspective view of the apparatus of FIG. 3 with the mixer tank, impeller, and a minimum working volume of fluid removed for clarity.

FIG. 5 is a side view of the apparatus of FIG. 4.

FIG. 6 is an opposite side view of the apparatus of FIG. 4.

FIG. 7 is a top perspective view of the apparatus of FIG. 4 illustrating a multicomponent assembly according to an embodiment of the invention.

FIG. 8 is a bottom perspective view of the apparatus of FIG. 7.

FIG. 9 is an enlarged perspective view of a handle according to an embodiment of the invention.

FIG. 10 is an enlarged perspective view of a connector according to an embodiment of the invention.

FIG. 11 is a graphical illustration of an assembly method according an embodiment of the invention.

FIG. 12 is a top perspective view of the impeller of FIG. 3, and a minimum working volume of liquid achieved with the apparatus of FIG. 3.

FIG. 13 is a side view of the minimum working volume and impeller of FIG. 12.

FIG. 14 is an enlarged side view of the minimum working volume and impeller of FIG. 12.

FIG. 15 is a top perspective view of an apparatus for reducing a minimum working volume according to another embodiment of the invention.

FIG. 16 is a front perspective view of a mixer system tank cut away to illustrate the apparatus of FIG. 15 and a minimum working volume of fluid.

FIG. 17 is a top perspective view of the impeller of FIG. 16, and the minimum working volume of liquid achieved with the apparatus of FIG. 15

FIG. 18 is a side view of the minimum working volume and impeller of FIG. 17.

FIG. 19 is an enlarged side view of the minimum working volume and impeller of FIG. 17.

FIG. 20 is a first side view of the apparatus of FIG. 15.

FIG. 21 is an opposite side view of the apparatus of FIG. 15.

FIG. 22 is an enlarged perspective view of a handle of the apparatus of FIG. 15.

FIG. 23 is a top view of the apparatus of FIG. 15.

FIGS. 24A and 24B are views of a low-profile impeller particularly suitable for use with embodiments of the invention.

FIG. 25 is an enlarged perspective view of a vessel suitable for use with embodiments of the invention depicting an impeller in an interior volume of the vessel.

FIG. 26 is a simplified perspective view of an apparatus for reducing a minimum working volume according to another embodiment of the invention that illustrates an inflatable compartment in an inflated state;

FIG. 27 is a simplified perspective view of the apparatus of FIG. 26 depicting the inflatable compartment in a deflated state;

FIG. 28 is a simplified, partially sectioned, perspective view of an apparatus for reducing a minimum working volume according to yet another embodiment of the invention; and

FIG. 29 is an additional simplified, partially sectioned, perspective view of the apparatus of FIG. 28.

### DETAILED DESCRIPTION OF EMBODIMENTS

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, or a rigid container, as the case may be. The term “vessel” as used herein is intended to encompass mixer and bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems.

As used herein, the term “bag” means a flexible or semi-rigid container or vessel used, for example, as a bioreactor or mixer for the contents within. In this regard, embodiments of the invention are suitable for use with mixers, bioreactors, and other devices or systems for mixing or processing fluids.

“Stirred tank” as used herein refers to a tank, housing, or vessel of a mixer, bioreactor, or the like that is configured to receive a fluid, and mix, stir, or otherwise agitate the fluid. The term is not limited to single-use mixers/bioreactors and is intended to encompass reactors and mixers generally, including those used in biological, chemical, industrial, environmental, and other applications. The term is likewise not limited to a particular apparatus for, or method of, mixing, stirring, or otherwise agitating the fluid. The term includes, but is not limited to, tanks of various sizes and shapes, including rectangular, square, and cylindrical tanks.

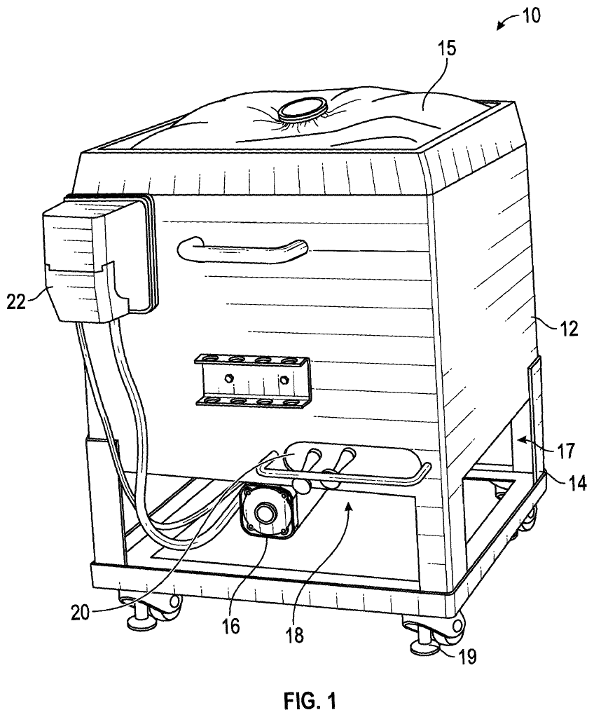

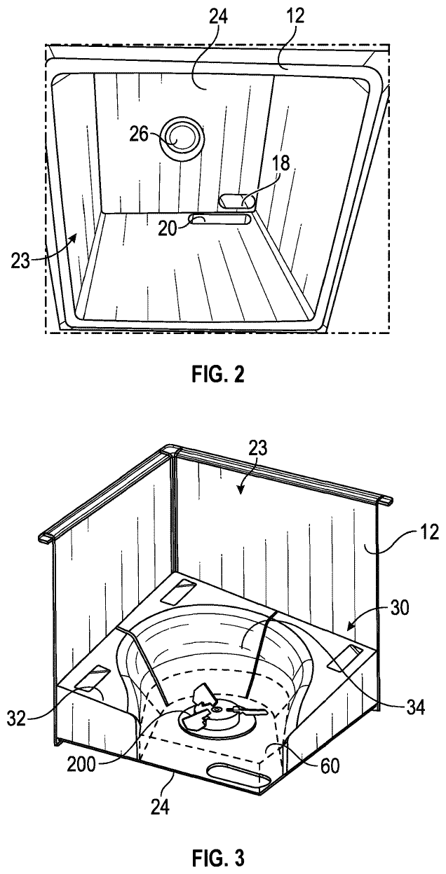

With reference to FIGS. 1 and 2, an exemplary mixer system 10 suitable for use with embodiments of the invention is illustrated. The mixer system 10 includes a generally rigid stirred tank 12 (referred to hereinafter simply as “tank 12”) mounted atop a base 14 that is movable via lockable casters 19. The base 14 has generally open sides 17 to allow a user to access an underside or bottom surface of the tank 12. The tank 12 may be formed, for example, from stainless steel, polymers, composites, glass, or other metals. As shown, in an embodiment, the tank 12 has a substantially rectangular shape. In other embodiments, the tank 12 may be a cylindrical bioreactor tank.

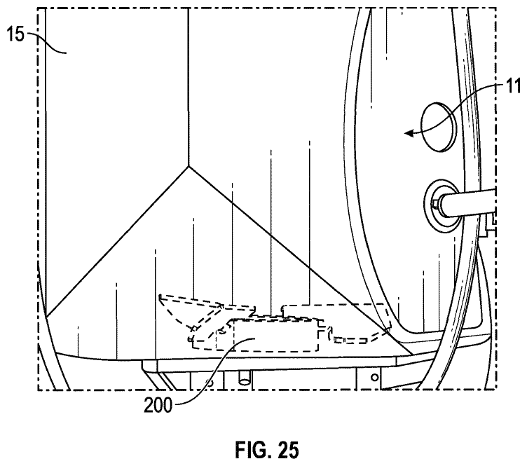

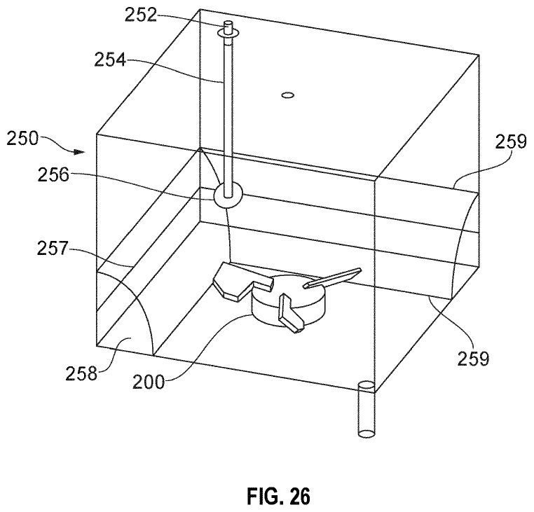

In operation, a single-use, flexible vessel or bag 15 is disposed within an interior 23 of the tank 12, which supports and protects the bag 15, among other functions. As will be appreciated, the bag 15 has an interior volume 11 (FIG. 25) that receives materials, e.g., a fluid, to be mixed/processed. To that end, the bag 15 includes an impeller 200 located within its interior volume 11. In embodiments, the impeller 200 includes one or more permanent magnets which allow it to be magnetically coupled to an external impeller drive motor 16.

As shown, the external impeller drive motor 16 is located outside of and underneath the tank 12 and may be accessed through the open sides 17 of the base 14. In embodiments, the drive motor 16 is secured to a bottom surface 24 of the interior 23 of the tank 12. The interior 23 also includes a drain aperture or hole 18, located on the bottom surface 24, which allows for the passage of a drain line from the bag 15 (FIG. 2). As will be appreciated, the drain 18 allows for selective removal of the contents of the bag 15.

The bottom surface 24 of the interior 23 further includes an impeller drive head 26 which, in embodiments, may be magnetically coupled to a magnet of a bag impeller as previously mentioned. The interior 23 also features a probe window 20 through which monitoring probes (e.g., pH, conductivity, temperature, etc.) may be inserted into the bag 15. Referring to FIG. 1, the mixer 10 includes a controller 22, which allows a user to monitor the probes and control operation of the mixer 10 including any pumps connected thereto.

Turning now to FIG. 3, a tank 12 is cut away to illustrate an apparatus 30 for reducing a minimum working volume according to an embodiment of the invention. The figure further depicts an impeller 200 and a reduced minimum working volume of fluid 60. A vessel or bag 15 has been omitted for clarity but, in an embodiment, would contain the impeller 200 and fluid 60.

The apparatus 30 includes a body 32 that, in use, sits on the bottom surface 24 of the interior 23 of the tank 12. The body 32 fits closely within the tank interior 23 and is generally configured, e.g., shaped, to match the shape of the tank interior 23. In the depicted embodiment, the body 23 is rectangular to match the rectangular mixer tank 12. The body 32 further includes a substantially arcuate U or C shaped interior portion 34 which partially surrounds the impeller 200 thereby reducing the minimum working volume 60 while allowing access to the drain 18 and probe window 20 (FIG. 2).

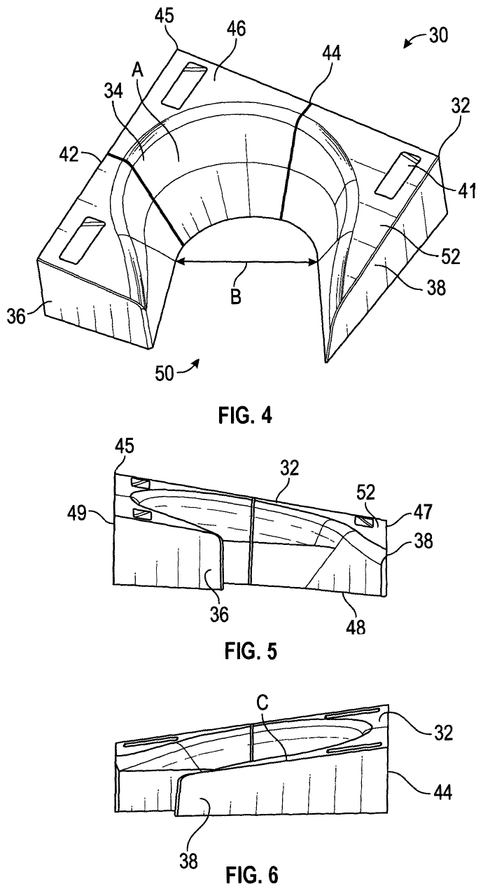

Referring now to FIGS. 4-8, the body 32 has an exterior profile that is shaped to closely fit within an interior of the stirred tank 12. In the depicted embodiment, the exterior profile of the body 32 is defined by four sides 36, 38, 42, 44, each of which are at a substantially 90-degree angle to adjacent sides, creating a generally quadrilateral shape. As will be appreciated, the exterior profile of the body 32 may vary in shape depending on the shape of the interior of the tank. For example, the body 32 may have a round or substantially circular exterior profile for use in known cylindrical reactor tanks.

The body 32 further includes an upper surface 46 and a bottom or lower surface 48 (FIG. 8). The interior portion 34 of the body extends through the body 32 from the upper surface 46 to the lower surface 48, creating an open body section that partially surrounds an impeller of a vessel (e.g., bag) and allows rotation of the same to mix/agitate a fluid in the vessel.

The body 32 includes an opening 50 that extends into the interior portion 34. In an embodiment, the opening 50 is formed by a void, cut away, or other absence of material in one or more sides 36, 38 of the body 32. The opening 50 is configured to facilitate removal of fluid from the bag 15 when the tank 12 is in use, and to be removed from the tank via the drain 18. In embodiments, the opening 50 allows access to the probe window 20 located on a front surface of the tank 12.

As shown in FIGS. 4-6, the interior portion 34 is a substantially continuous smooth surface. The surface is arcuate and is arranged in a general U or C shape. The surface is angled towards the opening 50 so that fluid 60 in the bag 15 within the interior portion 34 is directed out of the opening 50 toward the drain 18 of the tank 12. The angle of the surface A may vary, as can the shape, though a shape and angle directing fluid toward the opening 50 and drain 18 is desirable. In an embodiment, the angle A is approximately 30°.

In addition, in certain embodiments, a surface 52 of body 32 has an angle C of approximately 10°. This surface 52 is angled towards the opening 50 so fluid does not settle on the surface 52 during operation.

Though the surface of the interior portion 34 is shown as continuous and smooth, e.g., devoid of hard angles or flat portions, in certain embodiments it may have such surfaces. That said, a continuous smooth surface as depicted requires less fluid to submerge the impeller 200 and creates a lower minimum working volume 60 and helps to ensure that the interior portion 34 does not scratch or otherwise puncture the bag 15 in use.

In embodiments configured for use with 50 L, 100 L, and 200 L stirred tank mixers, the interior portion 34 has a base diameter B of 250 mm. In certain embodiments, a 25 mm space S between the terminal end of each impeller blade and the interior portion 34 (e.g., FIG. 12) is maintained. As will be appreciated, the diameter of the interior portion 32 may vary depending on the size and shape of the stirred tank 12, apparatus 30, and the length of the impeller blades.

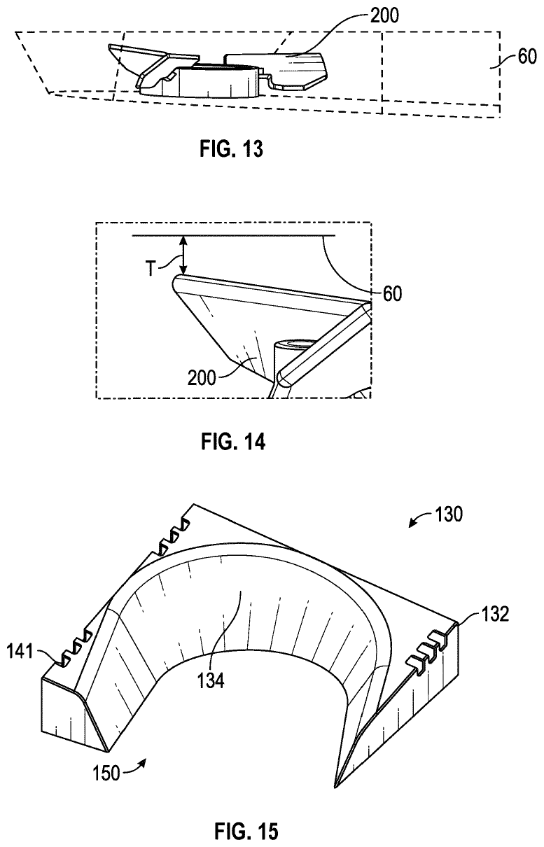

As shown in FIGS. 12-14, the impeller 200 is spaced apart from the interior portion 34 which defines the shape of the depicted minimum working volume of fluid 60. In particular, in an embodiment, the minimum working volume 60 was determined based on a 10 mm space between the highest point of the impeller 200 and the surface of the fluid 60.

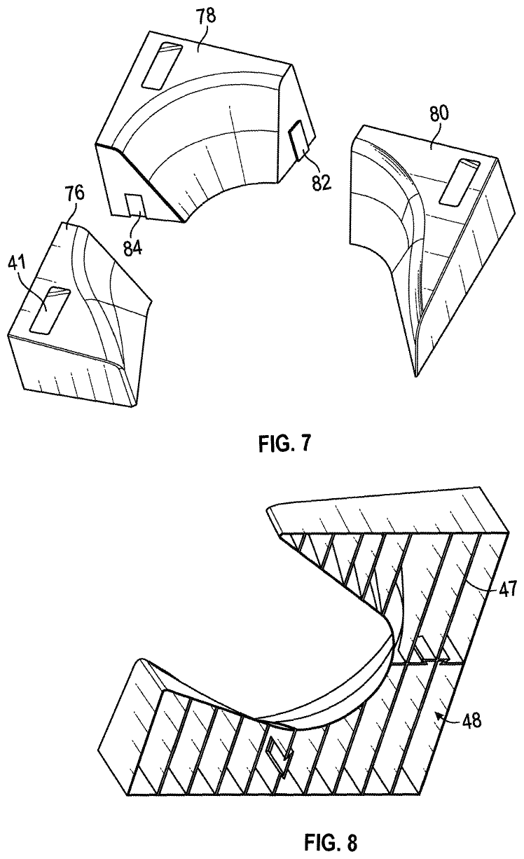

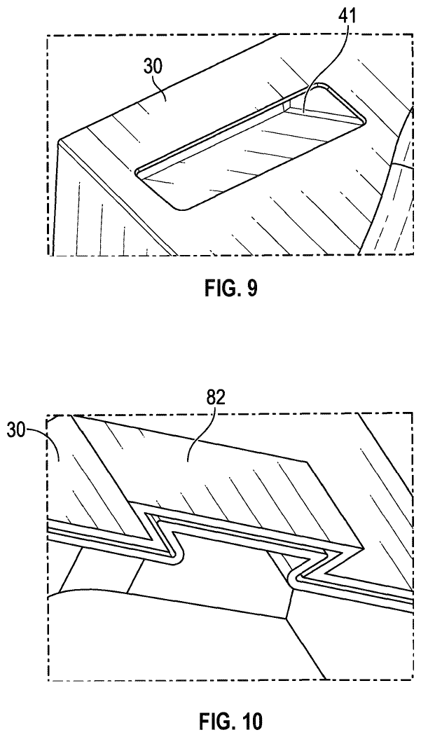

Referring now to FIGS. 5 and 6, in an embodiment, the body 32 has an overall wedge shape or profile. That is, the body 32 has a maximum height where sides 42 and 44 meet to form a high corner 45 that is directly across from the exposed corner of the tank 12 that contains the drain 18. These sides 42, 44 then taper down so that the other two corners 47 and 49 are lower than the high corner 45. The overall wedge shape and high corner 45 also function to direct fluid 60 in the bag 15 within the interior portion 34 toward the opening 50 and drain 18 in the tank 12.

In a specific embodiment configured for a 100 L stirred tank 12, the body 32 has a width of 498 mm and a length of 498 mm with a height of 186 mm. In an embodiment configured for use with a 50 L tank, the body 32 has a length and width of 388.7 mm and a height of 127 mm. For a 200 L tank, the length and width of the body 32 are 630 mm with a height of 275 mm. As will be appreciated, however, the external dimensions of the body 32 may vary depending upon the dimensions of the interior of the reactor/mixer tank.

Moreover, the shape of the body 32 is configured to accommodate a tank 12 that has a bottom surface 24 that is at a 2° angle toward the corner of the tank 12 with the drain 18. That is, the lower surface 48 of the body 32 may have a complementary angle so that the side walls 36, 38, 42, 44 sit flush with the walls of the tank 12.

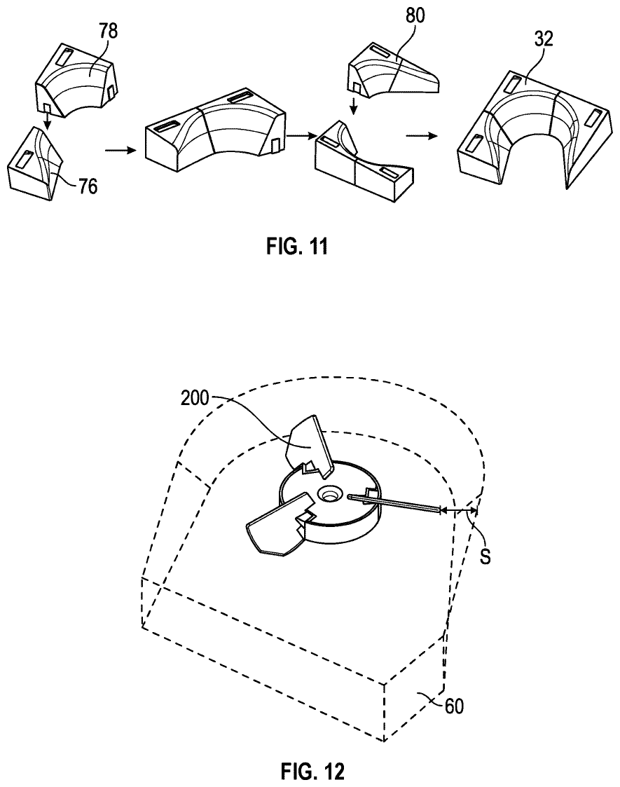

Referring now to FIGS. 7, 10, and 11, in embodiments, the body 32 may be an assembly of multiple selectively interconnectible body sections. In the depicted embodiment, the body 32 includes three such sections 76, 78, 80. The body sections 76, 78, 80 may be connected to one another via a connection mechanism that may include, for example, a protrusion 82 that slidably fits within a groove or channel 84 in an adjacent body section. As shown in FIG. 11, the body 32 may be assembled by slidably connecting each section 76, 78, 80 via the protrusion 82 and grooves 84. Various other connection mechanisms/methods may be employed without departing from the scope of the invention.

As will be appreciated, the body 32 can be broken down into a more easily transportable form via the selectively interconnectible body sections 76, 78, 80. This may be desirable for large mixers/bioreactors where the body 32 is likewise relatively large. However, in certain embodiments, the body 32 may include fewer or more than three sections. In certain other embodiments, the body 32 may include two sections and in other embodiments the body 32 may be unitary.

The body 32 may also have one or more handles 41 to facilitate placement of the body 32 in the tank 12, as well as removal from the tank 12. As shown, in certain embodiments, each body section 76, 78, 80 has its own handle 41, which may be a rectangular recess. In other embodiments, the handles may have a different shape/form and, in yet others, fewer (or no) handles may be present.

In certain 100 L and 200 L embodiments, the body 32 may include two handles that are 100 mm wide by 20 mm high and a third handle that is 75 mm wide by 16 mm high. A fourth handle may be 20 mm wide by 10 mm high. In a certain 50 L embodiment, the handles are 18 mm wide by 15 mm high. The exact dimensions (e.g., width and height) of the handles can vary depending on the size of the reactor/mixer tank, and are within the scope of the invention

As shown in FIG. 8, the body 32 may include a series of ribs 47. The ribs 47 are to strengthen and provide support to the body 32 when it is under load from fluid in the tank 12. As will be appreciated, in embodiments the body 32 may include greater or fewer ribs than depicted or may be solid.

In an embodiment, the apparatus 30 may be manufactured from a thermoplastic elastomer such as high-density polyethylene (HDPE). While HDPE may be suitable given its high strength to density ratio, other materials may be employed without departing from the invention. In certain embodiments, the apparatus 30 may be manufactured from nylon, polyurethane, or acrylonitrile butadiene styrene. The apparatus 30 may be vacuum cast, molded, or additively manufactured.

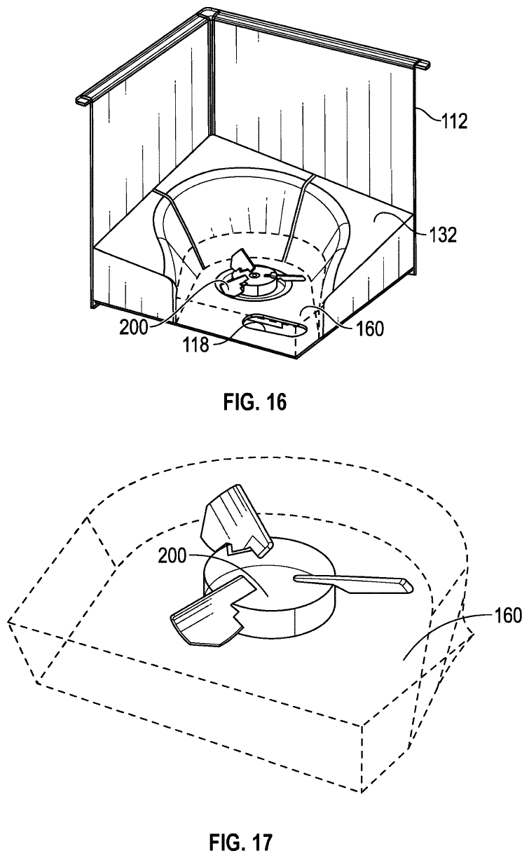

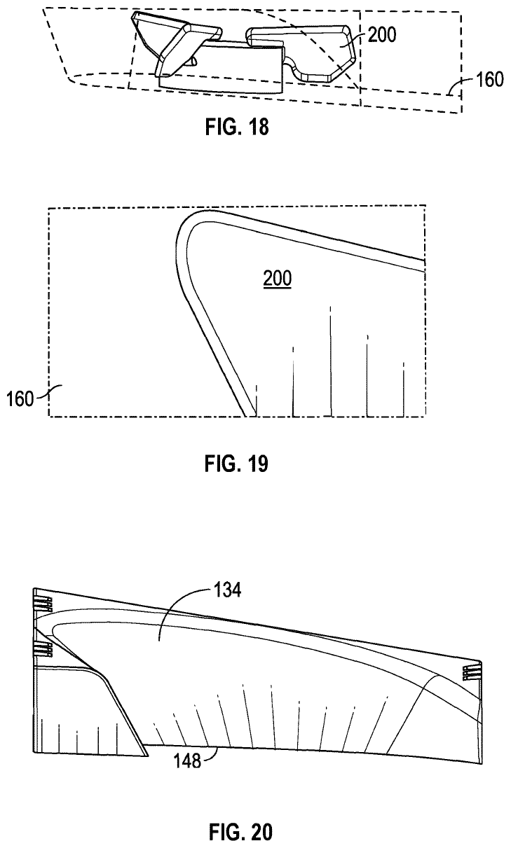

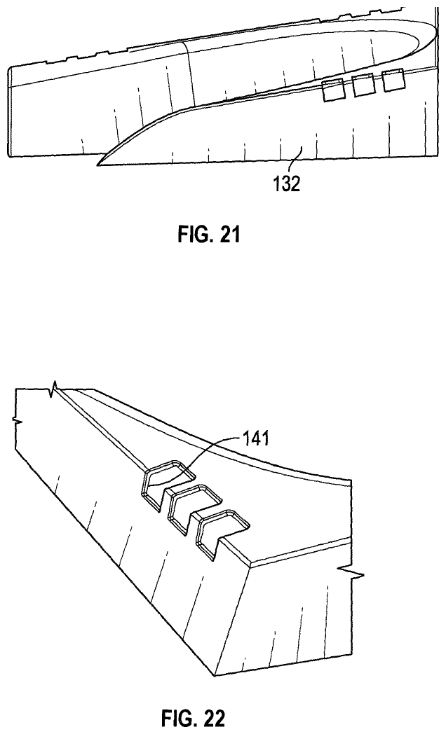

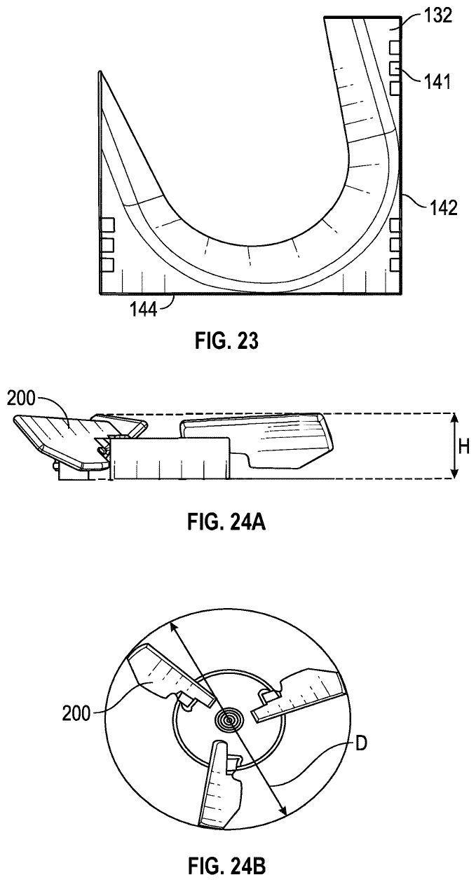

Referring now to FIGS. 15-23, an alternative embodiment of the inventive apparatus 130 is depicted. As shown, the apparatus 130 includes a body 132 featuring a substantially acuate U or C shaped interior portion 134 which partially surrounds the impeller 140 thereby reducing the minimum working volume 160 while allowing access to the drain 118. In this embodiment, the two depicted longer sides 142, 144 have a length of 388.7 mm.

This embodiment, which is configured for a smaller stirred tank 112 (e.g., 50 L), is unitary and features a series of notches 141 that serve as handles for placing and removing the apparatus 130 from the tank 112. When used in connection with impeller 200, the apparatus 130 has been able to achieve a 5.2 L minimum working volume in a 50 L stirred tank 112.

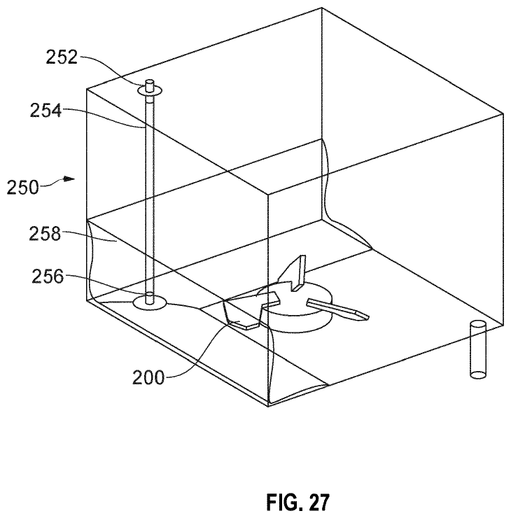

In further embodiments, illustrated by FIGS. 26-29, a reduced minimum working volume is achieved through modifications to the bag 15. In one example embodiment, shown in FIGS. 26 and 27, the bag 250 includes an inflatable compartment 258. The inflatable compartment 258 is constructed by welding a sheet of bag material 257 into the inside of the bag 250 around at least a portion of its bottom periphery. In the illustrated example, the bag material 257 is an elongated sheet, with the long edges thereof welded to the inside of the bag 250. The welds 259 can extend along a portion or all of the periphery of the bottom of the bag. The bag further includes a first barb 252 welded to the bag and configured to provide fluidic access to the inside of the bag. A first end of a tube 254 is connected to the first barb 252 and located inside the internal volume of the bag 250. A second barb 256 is welded to the inflatable compartment 258 and is connected to a second end of tube 254.

As illustrated, when the bag 250 is inflated a first weld 259 extends around at least a portion of the sidewall of the bag 250, and a second weld 259 extends along a bottom of the bag. This welding structure creates the inflatable compartment 258.

In order to inflate (see FIG. 26), the compartment 258 is inflated with a gas. The gas is pumped into a top barb 252 that is welded to a portion of the bag 250. The top barb 252 is fluidically connected to a tube 254 located on the inside of the bag 250. The tube 254 is further connected to a bottom barb 256 that is welded to the bag material 257 that creates the inflatable compartment 258. Conversely, to deflate (see FIG. 27), gas is pumped out of the inflatable compartment 258 through the barbs 252, 256 and tube 254.

Similar to the above embodiments, when the inflatable compartment 258 is in an inflated state (FIG. 26) the minimum working volume is reduced due to the fact that the compartment 258 takes up space at the bottom of the bag.

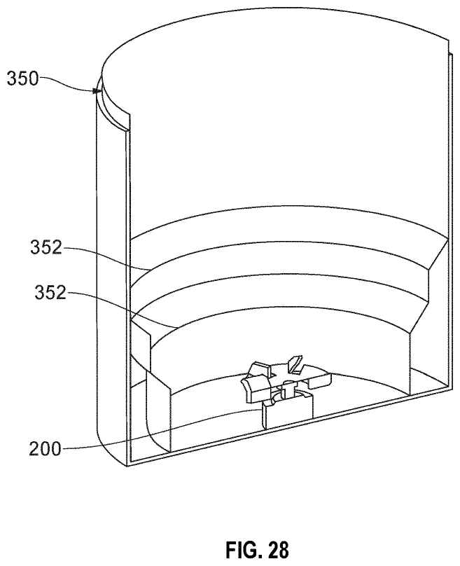

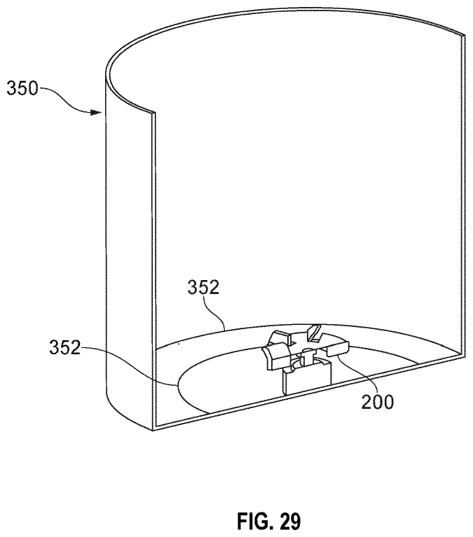

In a further example embodiment, as illustrated by FIGS. 28 and 29, bag 350 can be modified to have a varying cross-sectional area with one or more hinges/folds 352 along a length of the bag 350. In order to create a reduced minimum working volume, the hinges/folds 352 are unfolded (see FIG. 28). In an unfolded state, the minimum working volume is reduced because the cross-sectional area of the bag 350, at its base, is reduced. Conversely, the minimum working volume can be increased by collapsing/folding the hinges/folds 352, such that the bag 350 maintains a generally constant cross-sectional area (see FIG. 29).

Turning to FIGS. 24A and 24B, a low-profile impeller 200 may be utilized with the body 32. A traditional mixer impeller has a height of 108.3 mm. However, an impeller must be submerged in a fluid for effective mixing/agitation. As such, the lower the height of the impeller, the less fluid necessary to rise above that height. In embodiments, the low-profile impeller 200 may have a height H of approximately 39 mm (and an impeller diameter D of 220.5 mm).

By itself, such an impeller 200 provides a substantial reduction in minimum working volume from 32 L to 13.87 L in a 100 L stirred tank mixer. Significantly, the combination of an embodiment of the present apparatus 30 with impeller 200, results in a minimum working volume of 6.4 L in the same 100 L stirred tank. It was also found that impeller 200 provided comparable mixing/agitation performance when compared to taller impellers.

Impellers need not have a height of 39 mm to be effectively used with embodiments of the invention. It is contemplated that impellers having a height below that of approximately 108 mm may be sufficiently “low profile” to be used beneficially with the present apparatus 30. Of course, in embodiments, the apparatus 30 may be used with a conventional, taller mixer impeller and still achieve a significant reduction in minimum working volume.

In embodiments, the apparatus 30, 130 reduces minimum working volume and expedites mixing, i.e., the mixing the minimum working volume with the body 32, 132 in place takes less time than mixing the minimum working volume without the body 32, 132. In a specific embodiment, where minimum working volume with body 32, 132 and impeller 200 was 6.4 L, the mixing time was found to be 15 seconds vs 20 seconds without the apparatus 30, 130.

In certain embodiments, such as those that include a low-profile impeller 200, very high turndown ratios may be achieved.

Aspects of the invention also include a method of reducing a minimum working volume in a stirred tank 12 includes placing a body 32, 132 within an interior of the stirred tank 12. As described above, the body 32, 132 has upper 46 and lower surfaces 48 and an interior portion 34 extending through the body 32, 132, the interior portion 34 configured to receive and partially surround an impeller 200 of a vessel placed within the stirred tank 12, while allowing rotation of the impeller 200 to agitate fluid 60 in the stirred tank 12. The body 32, 132 further including an opening 50 in a side surface of the body 12, the opening 50 extending into the interior portion 34 and configured to allow fluid 60 to partially exit the interior portion when in use, and to be removed from the vessel via a drain 18 in the stirred tank 12.

Similarly, embodiments include a method of increasing a minimum working volume 60 in a stirred tank 12 removing a body 32, 132 from within an interior of the stirred tank 12. As discussed, the body 32, 132 has upper 46 and lower surfaces 48 and an interior portion 34 extending through the body 32, 132, the interior portion 34 configured to receive and partially surround an impeller 200 of a vessel placed within the stirred tank 12, while allowing rotation of the impeller 200 to agitate fluid 60 in the vessel. The body 32, 132 further including an opening 50 in a side surface of the body 12, the opening 50 extending into the interior portion 34 and configured to allow fluid 60 to partially exit the interior portion when in use, and to be removed from the vessel via a drain 18 in the stirred tank 12.

As used herein, an element or step recited in the singular and proceeded with the word “a” or “an” should be understood as not excluding plural of said elements or steps, unless such exclusion is explicitly stated. Furthermore, references to “an embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

Moreover, in the following claims, terms such as “first,” “second,” “upper,” “lower,” “bottom,” “top,” etc. are used merely as labels, and are not intended to impose numerical or positional requirements on their objects. Further, the limitations of the following claims are not written in means-plus-function format and are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. An apparatus for reducing a minimum working volume of a stirred tank comprising:
a body having upper and lower surfaces, and an exterior profile shaped to closely fit within an interior of the stirred tank, the body comprising:
an interior portion extending through the body from the upper surface to the lower surface, the interior portion configured to receive and partially surround an impeller within the stirred tank, while allowing rotation of the impeller to agitate a fluid in a vessel when in use; and
an opening in a side surface of the body, the opening extending into the interior portion and configured to facilitate removal of fluid from the vessel when the stirred tank is in use, and to be removed from the stirred tank via a drain.

2. The apparatus of claim 1, wherein the impeller is located within an interior volume of the vessel in the stirred tank.

3. The apparatus of claim 1, wherein the impeller is a low-profile impeller.

4. The apparatus of claim 1, wherein the interior portion has a substantially arcuate U-shape.

5. The apparatus of claim 1, wherein the exterior profile of the body is defined by four sides each at a substantially 90-degree angle to adjacent sides.

6. The apparatus of claim 5, wherein the four sides converge to form three corners where one of the corners has a height that is greater than heights of the other two corners, such that fluid in the vessel in the interior portion is directed toward the opening.

7. The apparatus of claim 1, wherein the body comprises a plurality of selectively interconnectible body sections.

8. The apparatus of claim 1, wherein the body includes at least one handle.

9. The apparatus of claim 1, wherein the interior portion comprises a substantially continuous smooth surface.

10. The apparatus of claim 1, wherein the stirred tank is a mixer or bioreactor.

11. The apparatus of claim 2, wherein the vessel is a collapsible bag or a bioreactor bag.

12. The apparatus of claim 1, wherein the body is manufactured from high-density polyethylene.

13. The apparatus of claim 1, wherein the exterior profile of the body is substantially circular.

14. An apparatus for reducing a minimum working volume of a stirred tank comprising:
a bag configured to fit within the stirred tank, the bag comprising:
a first barb welded to the bag configured to provide fluidic access to the inside of the bag;
a tube connected to the first barb and located inside an internal volume of the bag;
an inflatable compartment located inside and along a periphery of a bottom of the bag;
a second barb welded to the inflatable compartment and connected to the tube.

15. An apparatus for reducing a minimum working volume of a stirred tank comprising:
a bag configured to fit within the stirred tank, the bag comprising:
a plurality of folds or hinges along a length of the bag,
wherein when the folds or hinges are in an unfolded or unhinged state, the bag has a reduced cross-sectional area at its base.
